SEQUENCE LISTING (2 sequences)

SEQ ID NO: 1 - protein, 120 aa, Artificial Sequence
  DRalpha1-MOG35-55 polypeptide
MEVGWYRSPFSRVVHLYRNGKGGGGSLVPRGSGGGGIKEEHVIIQAEFYLNPDQSGEFMFDFDGDEIFHVDMAKKETVWRLEEFGRFASFEAQGALANIAVDKANLEIMTKRSNYTPITN

SEQ ID NO: 2 - DNA, 363 bp, Artificial Sequence
  DRalpha1-MOG-35-55 nucleic acid
atggaagttggttggtaccgttccccgttctcccgtgttgttcacctgtaccgtaacggtaaaggaggtggaggctcactagtgccccgaggctctggaggtggaggcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaattaa

(19) United States
(12) Patent Application Publication
(10) Pub. No.: US 2018/0271935 A1
(43) Pub. Date: Sep. 27, 2018

(54) DRALPHA1-MOG-35-55 TREATMENT OF TRAUMATIC BRAIN INJURY

(71) Applicants: Halina Offner, Portland, OR (US); Arthur Vandenbark, Portland, OR (US)

(72) Inventors: Halina Offner, Portland, OR (US); Arthur Vandenbark, Portland, OR (US)

(73) Assignee: OREGON HEALTH & SCIENCE UNIVERSITY, PORTLAND, OR (US)

(21) Appl. No.: 15/921,354

(22) Filed: Mar. 14, 2018

Related U.S. Application Data
(60) Provisional application No. 62/471,829, filed on Mar. 15, 2017.

(51) Int. Cl. A61K 38/16; A61P 25/00

(52) U.S. Cl. CPC A61K 38/16; A61P 25/00

(57) ABSTRACT

Methods and compositions used in treating traumatic brain injury using a recombinant DRα-MOG-35-55 construct are disclosed. The disclosed methods involve administering a pharmaceutical composition comprising DRα-MOG-35-55 and a pharmaceutically acceptable carrier to a subject that has had a traumatic brain injury.

### ACKNOWLEDGMENT OF GOVERNMENT SUPPORT

This invention was made with government support under grant numbers RO1 NS76013 and RO1 NS075887 awarded by the National Institutes of Health, and Merit Review Grant number I01 BX000226-07 awarded by the Department of Veterans Affairs. The government has certain rights in the invention.

### FIELD

This disclosure relates methods of treating traumatic brain injury with biological agents, particularly methods of treating traumatic brain injury with partial MHC Class II molecules.

### BACKGROUND

Traumatic brain injury (TBI) is a leading cause of mortality and morbidity with a lack of effective treatments. TBI can often result in long-term physical and cognitive deficits that involve primary and secondary injuries. The primary injury occurs at the moment of TBI, characterized by the disruption of blood brain barrier and blood vessels that contribute to brain edema (McKee and Lukens, Front. Immunol. 7:556, 2016; Pop and Badaut, Transl. Stroke Res. 2:533, 2011). This primary injury precedes downstream events contributing to a secondary injury cascade that includes the activation of brain-resident microglia and astrocytes and recruitment of peripheral immune cells into the brain (Kumar and Loane, Brain, Behavior, and Immunity 26:1191, 2012; McKee and Lukens, 2016; Raghupathi et al., Brain Pathol. 14:215, 2004; Ramlackhansingh et al., Ann. Neurol. 70:374, 2011). The inflammatory responses may start minutes after TBI onset and persist for weeks to months. As a result, these immune components may promote cell death during the early phase after TBI impact and contribute to subsequent neurological impairments during the later stage.

### SUMMARY

Targeting inflammation after TBI may serve as a promising strategy for the development of therapy. Disclosed herein are methods of treating TBI in a subject that involve administering to the subject an effective amount of a pharmaceutical composition comprising DRα1-MOG-35-55, alone or in combination with another active composition in a pharmaceutically acceptable carrier. In some examples, the method includes administering the composition after the onset of TBI. In other examples, the method includes administering to the subject one of more doses of DRα1-MOG-35-55 between 1-100 mg/kg. In still other examples, the composition may be administered by subcutaneous or intravenous administration

The foregoing and other features of the disclosure will become more apparent from the following detailed description, which proceeds with reference to the accompanying figures.

### BRIEF DESCRIPTION OF THE DRAWINGS

FIGS. 1A-1C is a series of panels showing that DRα1-MOG-35-55 treatment reduces neurodeficits after TBI. TBI was induced in C57BL/6 mice by FPI. Immediately after FPI, mice received subcutaneous injections of DRα1-MOG-35-55 (100 μg in 0.1 ml) or Vehicle and treatment was continued daily until the end of experiment (Day 10). FIG. 1A is a schematic illustrating the regimen of DRα1-MOG-35-55 administration and experimental design. Neurological assessments were performed to evaluate the motor, sensory and balance functions at indicated time points after TBI in DRα1-MOG-35-55 vs. Vehicle treated mice. n=10 per group. FIG. 1B shows modified Neurological Severity Score (mNSS) and FIG. 1C shows corner turning test. Data are presented as mean±SEM. *p<0.05, **p<0.01.

FIGS. 2A and 2B are a pair of panels showing that DRα1-MOG-35-55 treatment reduces lesion size after TBI. TBI was induced in C57BL/6 mice by FPI. Immediately after FPI, mice received subcutaneous injections of DRα1-MOG-35-55 (100 μg in FIG. 2A is a series of H&E stained images showing lesion area in coronal brain tissue sections of mice receiving Vehicle or DRα1-MOG-35-55 after TBI. FIG. 2B is a graph showing quantification of lesion size at indicated time points after TBI in mice receiving Vehicle or DRα1-MOG-35-55 treatment. *P<0.05, **P<0.01. n=8/group. Data are presented as mean±SEM.

FIGS. 3A-3E are a series of panels showing that DRα1-MOG-35-55 treatment reduces activation of CD11b+CD45hi cells in brain after TBI. TBI was induced in C57BL/6 mice by FPI. Immediately after FPI, mice received a subcutaneous injection of DRα1-MOG-35-55 (100 μg in 0.1 ml) or Vehicle. Treatment was continued daily until day 3 after TBI. FIG. 3A is a series of panels showing the flow gating strategy for microglia (CD11b⁺CD45^{int}), brain-infiltrating macrophages/activated microglia (CD11b⁺CD45^{hi}), and their surface expression of CD74, CD86 and CD206 at day 3 after TBI. FIG. 3B is a bar graph showing total numbers of microglia and brain-infiltrating macrophages per brain at day 3 after TBI in mice receiving DRα1-MOG-35-55 or Vehicle. FIG. 3C is a bar graph showing total numbers of microglia or brain-infiltrating macrophages per brain at day 3 after TBI in mice receiving DRα1-MOG-35-55 or Vehicle. FIG. 3D is a bar graph showing the expression of CD74 on microglia or brain-infiltrating macrophages at day 3 after TBI in mice receiving indicated treatments. In B-D, n=10 for Vehicle group; n=11 for DRα1-MOG-35-55 group. FIG. 3 E is a bar graph showing the expression of CD86 or CD206 in microglia at day 3 after TBI in mice receiving indicated treatments. n=7 mice per group. *p<0.05, **p<0.01. Data are presented as mean±SEM

FIGS. 4A-4D are a series of panels showing immune cell subsets in spleens of mice receiving DRα1-MOG-35-55 after TBI. TBI was induced in C57BL/6 mice by FPI. Immediately after FPI, mice received a subcutaneous injection of DRα1-MOG-35-55 (100 μg in 0.1 ml) or Vehicle. Treatment was continued daily until day 3 after TBI. FIG. 4A is a series of panels showing the expression of CD74, CD86 and CD206 in CD11b⁺ cells obtained from spleen. FIG. 4B is a bar graph showing the total numbers of splenocytes at day 3 after TBI in mice receiving DRα1-MOG-35-55 or Vehicle. FIG. 4C is a bar graph showing the numbers of CD11b⁺ cells obtained from spleen at day 3 after TBI in mice receiving DRα1-MOG-35-55 or Vehicle. FIG. 4D is a bar graph showing the numbers of CD11b⁺ cells expressing CD74, CD86 or CD206 obtained from spleen at day 3 after TBI in mice receiving DRα1-MOG-35-55 or Vehicle. n=4-5 mice per group. Data are presented as mean±SEM

FIGS. 5A-5D is a series of panels showing assessment of immune cell subsets in blood of mice receiving DRα1-MOG-35-55 after TBI. TBI was induced in C57BL/6 mice by fluid percussion injury (FPI). Immediately after FPI, mice received a subcutaneous injection of DRα1-MOG-35-55 (100 μg in 0.1 ml) or Vehicle. Treatment was continued daily until day 3 after FPI. FIG. 5A is the gating strategy showing the expression of CD74 on CD11b⁺ cells in peripheral blood. FIG. 5B is a bar graph showing the total number of blood cells at day 3 after FPI in mice receiving RTL DRα1-MOG or Vehicle. FIG. 5C is a bar graph shows the numbers of circulating CD11b⁺ cells at day 3 after FPI in mice receiving DRα1-MOG-35-55 or Vehicle. FIG. 5D is a bar graph showing the number of circulating CD11b⁺ cells expressing CD74 at day 3 after FPI in mice receiving DRα1-MOG-35-55 or Vehicle. *p<0.05, **p<0.01. n=9 per group. Data are presented as mean±SEM

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is a protein sequence of an exemplary human DRα1 polypeptide comprising a murine MOG-35-55 peptide and a linker, wherein the linker is covalently linked to the N-terminus of the DRα1 polypeptide and the MOG-35-55 peptide is linked to the N-terminus of the linker.

SEQ ID NO: 2 is an exemplary nucleic acid sequence encoding the protein of SEQ ID NO: 1.

### DETAILED DESCRIPTION

The human leukocyte antigen (HLA)-DRα1 domain linked to mouse (m)MOG-35-55 peptide (DRα1-MOG-35-55) is a partial major histocompatibility complex (MHC) class II construct that can inhibit neuroantigen-specific T cells and block the binding of the cytokine/chemokine, macrophage migration inhibitory factor (MIF) to its CD74 receptor on monocytes and macrophages (Benedek et al., Neurochem. Int. doi:10.1016/j.neuint.2016.10.007; Meza-Romero et al., J. Immunol. 192:4164, 2014; Wang et al., Transl. Stroke Res. 10.1007/s12975-016-0514-2). Recent studies have demonstrated the benefit of DRα1-MOG-35-55 in animal models of multiple sclerosis (experimental autoimmune encephalomyeliltis, EAE) and ischemic stroke (middle cerebral artery occlusion, MCAO).

As disclosed herein, DRα1-MOG-35-55 significantly enhances functional outcomes, reduces brain lesion size, blocks infiltration of CD11b⁺ cells into the injured brain, and promotes an anti-inflammatory phenotype in activated CD11b⁺CD45^{hi} cells that infiltrate from the periphery or that result from local activation of resident CD11b⁺CD45^{int} microglia after TBI. These changes observed in the injured brain were in stark contrast with increased numbers of activated monocytes in the blood and the lack of any demonstrable changes in the spleen.

Recombinant T-cell receptor (TCR) ligands (RTL) are molecular constructs comprised of covalently linked al and (31 domains of MHC II molecules with an attached antigenic peptide (Pan et al., Transl. Stroke Res. 5:577, 2014; Zhu et al., Transl. Stroke Res. 5:612, 2014; Zhu et al., Transl. Stroke Res. 6:60, 2015). Unlike four-domain MHC II molecules that induce T cell activation, RTLs are partial agonists of TCR and can deviate autoreactive T cell responses towards an anti-inflammatory phenotype (Burrows et al., J. Immunol. 167:4386, 2001; Wang et al., J. Immunol. 171:1934, 2003). Moreover, RTL constructs can block binding and downstream signaling of the cytokine/chemokine, macrophage migration inhibitory factor (MIF) through its CD74 receptor on monocytes and macrophages (Vandenbark et al., J. Autoimmun. 40:96, 2013). It was previously demonstrated that neurodegeneration after TBI is dependent upon CD74 expression on antigen presenting cells (Tobin et al., Acta Neuropathol. Commun. 2:143, 2014). RTL treatment of stroke has been shown to inhibit brain-reactive T cells without inducing general immunosuppression (Dziennis et al., Metab. Brain Dis. 26:123, 2011). Previous studies demonstrated that RTL treatment can protect against EAE and ischemic brain injury in mice (Dziennis et al., 2011; Pan et al., 2014; Subramanian et al., Stroke 40:2539. 2009; Zhu et al., 2014; Zhu et al., 2015). The clinical translation of RTL for treatment of acute thrombolytic or TBI events is restricted by the requirement for rapid matching of recipient MHC II with the (31 domains of the RTL construct. The MHC construct DRα1-MOG-35-55 utilized in the methods disclosed herein does not include an MHC II β1 domain. Because the DRα1 domain is expressed in all humans, DRα1-MOG-35-55 treatment does not require HLA screening of potential recipients and could be administered immediately after TBI in human subjects.

Our current data in TBI indicate minimal impact of DRα1-MOG-35-55 on CD11b⁺ cells in spleen, but a near doubling of total cells and CD74-expressing CD11b⁺ cells in blood. In contrast, DRα1-MOG-35-55 treatment of TBI strongly inhibits the activation and recruitment of brain-infiltrating CD11b⁺CD45^{hi} cells and their expression of CD74, as well as reduced expression of the co-stimulatory CD86 molecule but enhanced expression of the anti-inflammatory M2 marker, CD206, on CD11b⁺CD45^{int} microglial cells within the CNS. These findings suggest that DRα1-MOG-35-55 binding to CD74 resulting in blockade of MIF activity that prevents activation and recruitment of circulating CD11b⁺ cells into the injured brain after TBI and enhancement of M2-like microglial activity within the CNS.

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Administration: To provide or give a subject an agent, such as a pharmaceutical composition comprising DRα1-MOG-35-55 by any effective route. Exemplary routes of administration include, but are not limited to parenteral injection, such as intravenous, subcutaneous, or intraperitoneal injection.

Effective amount: An amount of agent, such as DRα1-MOG-35-55, that is sufficient to generate a desired response, such as the reduction or elimination of a sign or symptom of a condition, such as TBI. Alternatively, an effective amount may be an amount sufficient to generate a desired response in a cell or cell type, such as an effective amount to protect a neuron or other cell of the nervous system from damage resulting from TBI.

When administered to a subject, a dosage will generally be used that will achieve target tissue concentrations that have been shown to achieve activity in vitro. In some examples, an “effective amount” is one that prophylactically treats one or more symptoms and/or underlying causes of a disorder or disease. An effective amount can also be an amount that therapeutically treats one or more symptoms and/or underlying causes of a disorder or disease.

Treating a disease: Inhibiting the full development of a disease or condition, for example, in a subject who has a condition such as TBI. Treatment refers to any therapeutic intervention that ameliorates a sign or symptom of a disease or pathological condition. The term “ameliorating,” with reference to a disease or pathological condition, refers to any observable beneficial effect provided by a pharmaceutical composition. The beneficial effect can be evidenced, for example, by a delayed onset of clinical symptoms of the disease in a susceptible subject, a reduction in severity of some or all clinical symptoms of the disease, a slower progression of the disease or by other clinical or physiological parameters associated with a particular disease. A “prophylactic” treatment is a treatment administered to a subject who does not exhibit signs of a disease or exhibits only early signs for the purpose of decreasing the risk of developing pathology. A therapeutic treatment is a treatment administered to a subject who has already exhibited signs or symptoms of a disease.

Subject: A living multicellular vertebrate organism, a category that includes, for example, mammals and birds. A “mammal” includes both human and non-human primates (such as monkeys), as well as non-primate mammals such as mice or rabbits any other research animals. In some examples, a subject is a human patient, such as a patient that has had or is at risk of developing an ischemic event.

Traumatic brain injury (TBI): An injury to the brain resulting from an external force, such as a blow or jolt to the head or a penetrating head injury. TBI occurs due to a sudden acceleration or deceleration with the cranium or a combination of movement and sudden impact. Damage occurs both at the time of injury, as well as minutes to days later, for example, due to changes in blood flow and pressure within the cranium. TBI is classified from mild (including concussion) to severe. Injuries that may be associated with TBI include those resulting from Closed Head Injuries, wherein the skull remains intact with no penetration; Open Head Injuries in which the skull is penetrated; Diffuse Axonal Injuries, in which diffuse cellular injury to the brain results from rapid rotational movement; Contusions of the brain; Penetrating Trauma in which an object enters the brain; Secondary Injuries in which swelling and chemicals released promote cell injury or death (including intracranial hemorrhage, brain swelling, increase intercranial pressure, infection inside the skull, chemical changes leading to cell death, or increase fluid inside the skull; and Acquired Brain Injuries, such as those caused by anoxia and hypoxia, such as caused by breathing problems, cardiac arrest, bleeding, drugs or other poisons, including carbon monoxide poisoning.

### II. Pharmaceutical Compositions Comprising DRα1-MOG-35-55

The disclosed methods utilize pharmaceutical compositions including DRα1-MOG-35-55. DRα1-MOG-35-55 is a polypeptide which includes a DR1 MHC class II al domain or fragment thereof and does not include MHC class II α2, β1, or β2 domains, but does include an antigenic peptide (MOG-35-55) and a linker sequence. Exemplary DRα1 domains and MOG-35-55 peptides are disclosed in International Patent Publication WO 2015/051330, incorporated herein by reference. In some embodiments, DRα1-MOG-35-55 includes a mouse or human MOG-35-55 peptide (e.g., amino acids 35-55 of mouse or human myelin oligodendrocyte glycoprotein). One example of DRα1-MOG-355-55 is a polypeptide including or consisting of, or having a sequence at least 75%, 80% 85%, 90%, 95%, 96%, 97%, 98% or 99% identical to, the following sequence:

(SEQ ID NO: 1)
MEVGWYRSPFSRVVHLYRNGKGGGGSLVPRGSGGGGIKEEHVIIQAE
FYLNPDQSGEFMFDFDGDEIFHVDMAKKETVWRLEEFGRFASFEAQG
ALANIAVDKANLEIMTKRSNYTPITN

In some examples, the DRα1-MOG-35-55 polypeptide is encoded by a nucleic acid sequence including or consisting of, or having a sequence at least 80%, 85%, 90%, 95%, 96%, 97%, 98%, or 99% identical to, the following sequence:

(SEQ ID NO: 2)
ATGGAAGTTGGTTGGTACCGTTCCCCGTTCTCCCGTGTTGTTCACCT
GTACCGTAACGGTAAAGGAGGTGGAGGCTCACTAGTGCCCCGAGGCT
CTGGAGGTGGAGGCATCAAAGAAGAACATGTGATCATCCAGGCCGAG
TTCTATCTGAATCCTGACCAATCAGGCGAGTTTATGTTTGACTTTGA
TGGTGATGAGATTTTCCATGTGGATATGGCAAAGAAGGAGACGGTCT
GGCGGCTTGAAGAATTTGGACGATTTGCCAGCTTTGAGGCTCAAGGT
GCATTGGCCAACATAGCTGTGGACAAAGCCAACTTGGAAATCATGAC
AAAGCGCTCCAACTATACTCCGATCACCAATTAA

DRα1-MOG-35-55 (e.g., SEQ ID NO: 1) can be combined with a pharmaceutically acceptable carrier appropriate for the particular route of administration being employed. One of skill in the art in light of this disclosure would understand how to combine DRα1-MOG-35-55 with the appropriate carrier for use in a particular route of administration. Dosage forms of DRα1-MOG-35-55 include excipients recognized in the art of pharmaceutical compounding as being suitable for the preparation of dosage units as discussed below. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Compositions comprising DRα1-MOG-35-55 can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents can be biologically inactive and can be administered to patients without causing deleterious side effects or interactions with DRα1-MOG-35-55.

DRα1-MOG-35-55 can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Pharmaceutical compositions comprising DRα1-MOG-35-55 may be formulated for use in parenteral administration, e.g. intravenously, intramuscularly, subcutaneously or intraperitoneally, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers.

The parenteral preparations may be solutions, dispersions or emulsions suitable for such administration. Pharmaceutically parenteral formulations and ingredients thereof are sterile or readily sterilizable, biologically inert, and easily administered. Pharmaceutically acceptable carriers used in parenteral formulations comprising DRα1-MOG-35-55 of are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Injection solutions, emulsions and suspensions may be prepared from sterile powders, granules and tablets of the kind previously described. Unit dosage formulations are those containing a daily or other dose or unit, daily sub-dose, as described herein above, or an appropriate fraction thereof, of the active ingredient(s).

### III. Methods of Treating Traumatic Brain Injury

Disclosed herein are methods of treating TBI. The disclosed methods include administering an effective amount (such as a therapeutically effective amount) of a pharmaceutical composition including a DRα1-MOG-35-55 polypeptide to a subject having a TBI. In some examples, the pharmaceutical composition including a DRα1-MOG-35-55 polypeptide is administered to the subject within minutes of the TBI (e.g., within 5-60 minutes, such as within 5 minutes, 10 minutes, 15 minutes, 20 minutes, 30 minutes, 40 minutes, 50 minutes or 60 minutes of the TBI) or within hours of the TBI (e.g., within 1-24 hours, 4-18 hours, 6-12 hours, or 10-16 hours, such as within 1 hour, 2 hours, 3 hours, 4 hours, 6 hours, 8 hours, 10 hours, 12 hours, 16 hours, 18 hours, or 24 hours of the TBI). In other examples, the pharmaceutical composition including a DRα1-MOG-35-55 polypeptide is administered to the subject days or weeks after the TBI, such as after 1-7 days, 5-10 days, 7-14 days, 10-28 days, 21-50 days, or more, after the TBI.

Suitable routes of administration of DRα1-MOG-35-55 polypeptide include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods include, but are not limited to, intravenous, intramuscular, intraperitoneal, intraspinal, intrathecal, intracerebroventricular, intraarterial, intranasal, and subcutaneous injection.

Amounts and regimens for the administration of DRα1-MOG-35-55 to a subject can be determined by one of skill in the art. Typically, the dose range will be from about 0.1 μg/kg body weight to about 100 mg/kg body weight. Other suitable ranges include doses of from about 100 μg/kg to 10 mg/kg body weight or more (such as about 0.1-10 mg/kg, about 1-20 mg/kg, about 5-50 mg/kg, or about 10-100 mg/kg). In certain embodiments, the effective dosage will be selected within narrower ranges of, for example, 5-40 mg/kg, 10-35 mg/kg or 20-25 mg/kg. In other examples, the dosage is about 1-100 mg, such as about 1-10 mg, about 5-25 mg, about 10-50 mg, about 25-60 mg, or about 50-100 mg (for example, about 1 mg, 5, mg, 10 mg, 15 mg, 20 mg, 25 mg, 30 mg, 35 mg, 40 mg, 45 mg, 50 mg, 55 mg, 60 mg, 70 mg, 80 mg, 90 mg, or 100 mg). In particular examples, the dose is about 20-60 mg, and in one non-limiting example, about 25 mg.

These and other effective unit dosage amounts may be administered in a single dose, or in the form of multiple daily (for example, for 1-10 days or more), weekly, or monthly doses, for example in a dosing regimen comprising from 1 to 5 or 2-3 doses administered per day, per week, or per month. In some examples, the composition including the DRα1-MOG-35-55 polypeptide is administered daily for 1 or more days (for example, 1-10 days) and is followed by weekly or monthly boosts. The dosing schedule may vary depending on a number of clinical factors, such as the subject's clinical condition and/or sensitivity to the protein. One non-limiting example of a dosing schedule for TBI is 25 mg administered daily for 1, 2, 3, 4, 5, or more days following the TBI.

The methods of treatment herein may be used in combination with one or more courses of treatment conventionally used in the treatment of TBI, including the use of medications used to limit secondary damage to the brain. These include use antibiotics for possible infections (cefuroxime, ceftriaxone, metronidazole, vancomycin), diuretics to reduce tissue fluids (furosemide, bumetanide, torsemide, ethacrynic acid, hydrochlorthiazide, chlorthalidone, metolazone, spironolactone, eplerenone, triamterene, amiloride), anti-seizure/anti-convulsant/anti-epileptic medications (Dilantin, phenobarbital, Phenytoin, Fosphenytoin, Levetiracetam, valproate sodium); coma-inducing drugs (propofol, pentobarbital, thiopental); and calcium channel blockers (Nimodipine, Nicardipine, Amlodipine).

### EXAMPLES

The following examples are illustrative of disclosed methods. In light of this disclosure, those of skill in the art will recognize that variations of these examples and other examples of the disclosed method would be possible without undue experimentation.

### Example 1—Materials and Methods

Animals:

Male C57BL/6 mice (7-8 weeks old, 20-25 g body weight) were housed in animal facilities at Tianjin Neurological Institute (Tianjin, China) under standardized light-dark cycle, and provided with access to food and water. All animal experiments were performed in accordance with the institutional animal care guidelines approved by the Chinese Small Animal Protection Association. All animal studies were approved by the Animal Care and Use Committees of Tianjin Neurological Institute. Animals were randomly assigned to experimental groups. The experimental groups were as follows: FPI+ vehicle and FPI+DRα1-MOG-35-55.

Induction of Fluid Percussion Injury (FPI) in Mice:

The mice were anesthetized with 10% chloral hydrate (3 mg/kg, intraperitoneal injection), cleaned and shaved, then placed in a stereotaxic instrument with an attachment for mouse surgery (Stoelting, Inc., IL, USA). A 2 mm hole was drilled, with dura intact, in the skull over the left parietal cortex (antero-posterior: +1.5 mm; medio-lateral: −1.2 mm). A 12-16 ms FPI was delivered at a pressure of 1.3-1.5 atm. After injury, the scalp incision was closed with interrupted 4-0 silk sutures. After surgery, mice were treated immediately with Vehicle or DRα1-MOG-35-55 by subcutaneous injection and daily thereafter for 9 consecutive days in housing with a 12-h light-dark cycle with food and water.

DRα1-MOG-35-55 Cloning, Production and Purification:

Cloning, production, and purification of the DRα1-mMOG-35-55 construct have been described previously (Meza-Romero et al., J. Immunol. 192:4164, 2014). Briefly, DRα1-mMOG-35-55 was designed as a single gene joining the mouse (m)MOG-35-55-encoding DNA sequence upstream of the HLA-DRα1 domain with a flexible linker (containing a thrombin cleavage site) between both elements. This single exon was cloned between the NcoI and XhoI restriction sites of the pET21d(+) vector, expressed in Escherichia coli, and the protein purified following standard purification techniques including anion exchange and size exclusion chromatography in the presence of 6 M urea. Protein was refolded after extensive dialysis in 20 mM Tris, pH 8.5, then concentrated to 1 mg/ml and flash frozen.

DRα1-MOG-35-55 Administration:

After FPI surgery, mice were randomly divided into two groups, one receiving 0.1 ml (100 μg) DRα1-MOG-35-55, the other receiving 0.1 ml Vehicle (5% dextrose in Tris-HCl, pH 8.5) by subcutaneous injection. DRα1-MOG-35-55 or Vehicle was administrated by subcutaneous injection immediately after FPI and was given on 9 consecutive days until the experiment ended. Both Vehicle and DRα1-MOG-35-55 treated mice were evaluated for neurological deficits and lesion size using histopathological staining on days 1, 3, 7 and 10. On day 3 after FPI, brain, spleen and blood were harvested for analysis of immune cell counts.

Neurological Assessment:

Neurological deficits were assessed using the modified Neurological Severity Score (mNSS) and corner turning tests at day 1, day 3, day 7 and day 10 after FPI by at least two investigators blinded to the treatments of FPI mice in each experiment. The mNSS rates neurological functioning and includes a composite of motor, sensory, reflex and balance tests. The corner turning test was used to assess sensorimotor and postural asymmetries. Each mouse being tested was allowed to enter a corner with an angle of 30 degrees and was required to turn either to the left or the right to exit the corner. This was repeated and recorded 10 times, with at least 5 minutes between trials, and the percentage of left turns out of total turns was calculated.

Lesion Size Measurement:

Lesion volume was measured as previously described (Basrai et al., PLoS One 11:e0153418, 2016; Meng et al., PLoS One 9:e106238, 2014; Tobin et al., Acta Neuropathol. Commun. 2:143, 2014). At day 1, 3, 7 or 10 after surgery, mice were transcardially perfused with PBS, followed by 4% paraformaldehyde to fix the brain tissue. After embedding with paraffin, serial 6 μm thick sections were cut through the injury site (bregma −1.5 mm to 1.50 mm). For each mouse, every 20^{th} brain section (120 μm apart, ˜25 sections per brain) were collected for Hematoxylin and Eosin (H&E) staining. To determine lesion area of each slice from H&E, bright field images at 4× magnification were obtained of the lesion site, and two investigators blinded to the experiment calculated the lesion area by tracing damaged or abnormal looking tissue in the ipsilateral cortex using Image J 1.38 (National Institutes of Health, Bethesda, Md., USA). The down boundary of the lesion outline was determined by drawing the area in which the immune cell infiltration was observed, as previously described (Basrai et al., PLoS One 11:e0153418, 2016). For upper boundary of the lesion area, we outlined the expanse of the lesion using a mouse atlas (Tobin et al., Acta Neuropathol. Commun. 2:143, 2014; Tsukano et al., Sci. Rep. 6:22315, 2016). In order to confirm superficial lesion measurements using a digital caliper, we assessed the margins of the lesion in the anterior posterior plane. The lesion volumes were computed by integrating the lesion area of each slice measured at each coronal level and the distance between two sections.

Flow Cytometry of Brain, Spleen and Blood:

At day 3 after FPI, brains and spleens were harvested and homogenized with 40 μm nylon cell strainers (Becton Dickinson, Franklin Lakes, N.J., USA) in PBS. Cell suspensions were cetrifuged at 2,000 rpm for 5 min, and cell pellets were collected. Thereafter, 5 ml of 70% Percoll solution (GE Healthcare Bio Science AB, Uppsala, Sweden) were used to resuspend the brain cell pellet, and a 30% Percoll solution was overlaid. The gradient was centrifuged at 2,000 rpm for 30 min at room temperature. Single cells in the interface between 30% and 70% Percoll were collected for antibody staining. Cells were diluted to 1×10⁶ cells in 100 μl PBS solution with 1% BSA and stained for antibodies and isotype control. For splenocytes, erythrocytes were removed using 1×RBC lysis buffer (eBioscience, Inc. San Diego, Calif. USA) as indicated in the instruction manual. Briefly, the cell pellet was resuspended in 5 ml of 1×RBC lysis buffer per spleen and incubated for 5 min at room temperature. Thereafter, 30 ml 1×PBS were added to stop the reaction. After centrifugation, the cell pellet was resuspended in 1×PBS solution for cell staining. For blood samples, mononuclear cells were isolated from the whole-blood of angular vein specimens and stained with fluorescent-labeled antibodies. All antibodies were purchased from Biolegend (San Diego, Calif., USA) unless otherwise indicated, and the staining protocol followed the manual's instructions. The following antibodies for the flow experiment were used: CD45-Pecy7, CD11b-Percp, CD86-PE, CD206-FITC and CD74-APC. Fluorescence minus one (FMO) controls were stained, respectively. Flow cytometry data were obtained on C6 (BD Bioscience, San Jose, Calif., USA) and analyzed by Flow Jo version 7.6.1 (Informer Technologies, Walnut Creek, Calif., USA).

Statistical Analysis:

All data are shown as mean±SEM. Statistical analyses were performed using GraphPad 6.0 software. Two-tailed unpaired Student's t-test was used to determine significance of differences between two groups. Two-way ANOVA with multiple comparisons followed by the Bonferroni post hoc test were used for comparisons of multi-group data. P values <0.05 were considered significant.

### Example 2—DRα1-MOG-35-55 Attenuates Neurodeficits and Lesion Volume in FPI Mice

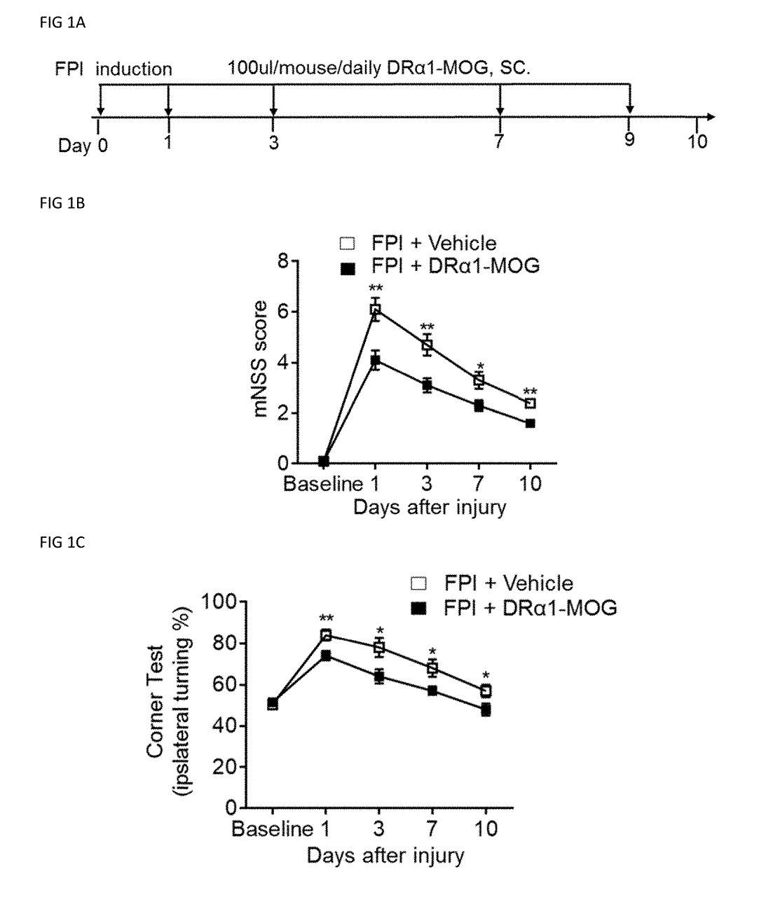

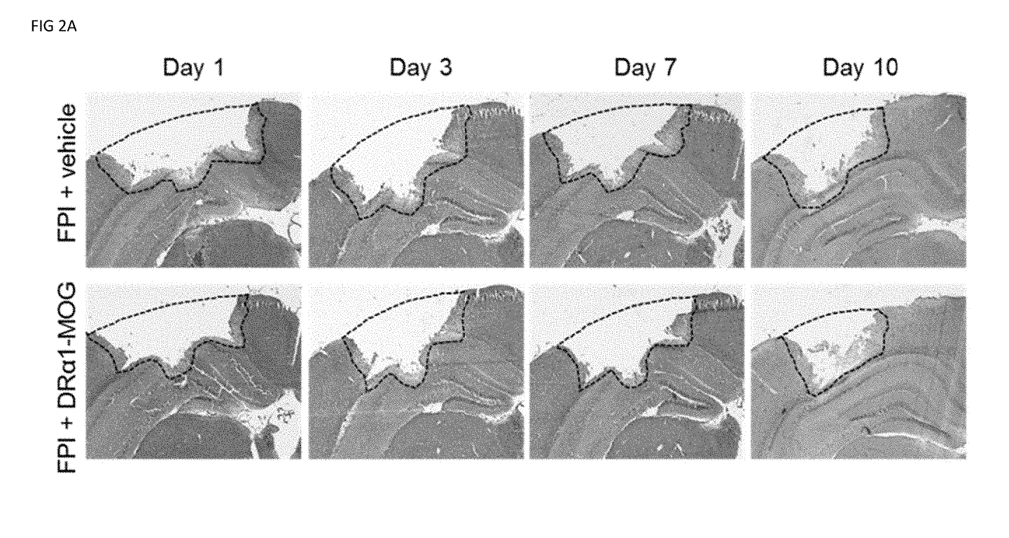

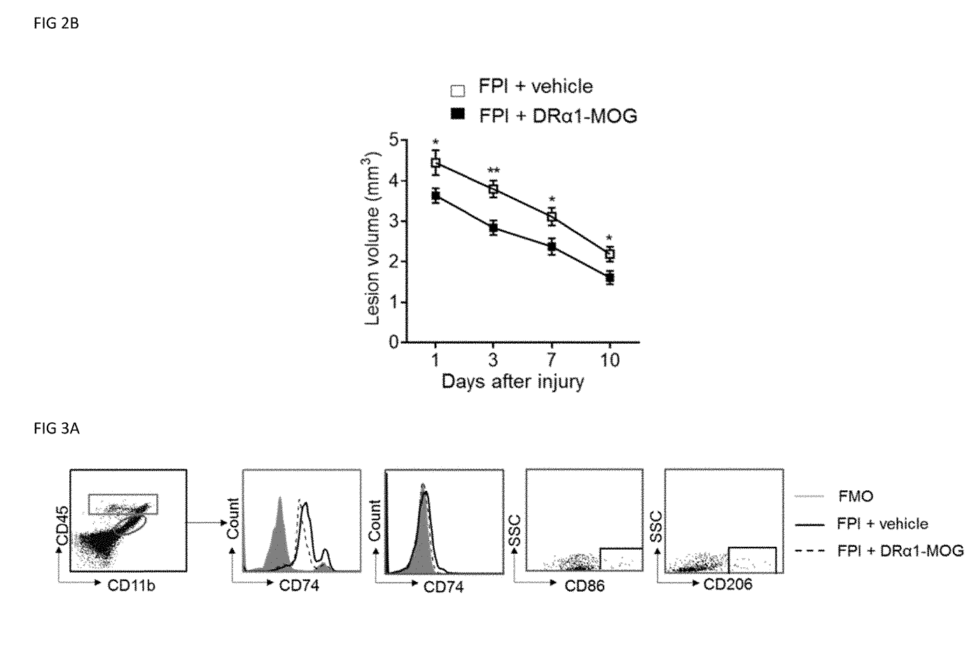

To assess the therapeutic effects of DRα1-MOG-35-55 after TBI, neurological impairment scores and lesion volumes in mice subjected to fluid percussion injury (FPI) were measured. Mice received nine daily s.c. injections of 100 μg DRα1-MOG-35-55 or vehicle starting immediately after FPI (FIG. 1A) and were assessed intermittently and 24 hours after the final treatment (day 10) using the modified Neurological Severity Score (mNSS) and the Corner Turning test. DRα1-MOG-35-55 treatment significantly reduced both neurodeficit measures (FIGS. 1B and 1C) as well as lesion volumes (FIGS. 2A and 2B) at all time points measured after TBI compared to vehicle controls.

### Example 2—DRα1-MOG-35-55 Reduces Brain Infiltration of CD11b⁺ Cells and their Expression of CD74

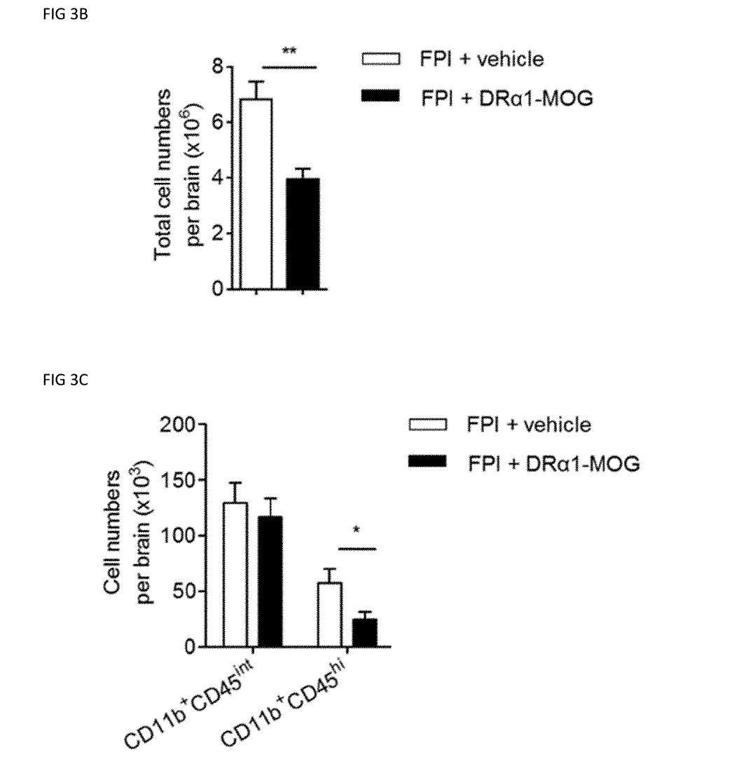

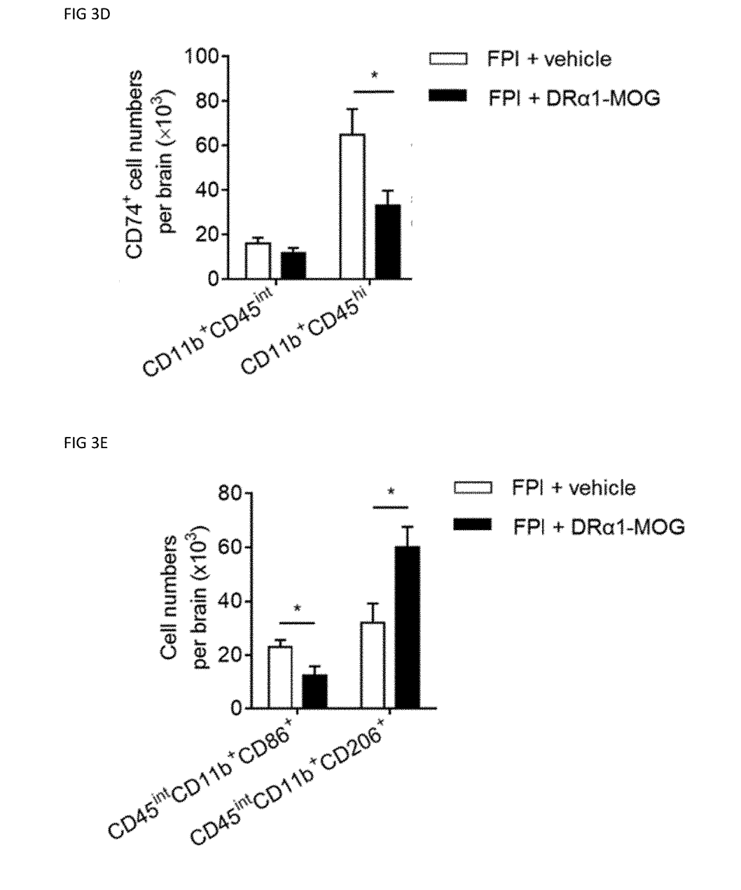

To determine whether DRα1-MOG-35-55 treatment of brain injury could affect the cell numbers and CD74 expression of CD11b⁺ cells in the brain, numbers of non-activated brain-intrinsic microglia (CD11b⁺CD45^{int}) and brain-infiltrating macrophages/activated microglia (CD11b⁺CD45^{hi}) after TBI were assessed. DRα1-MOG-35-55 treatment significantly reduced the total number of brain-infiltrating cells [vehicle: 6.83±0.64 versus DRα1-MOG-35-55: 3.95±0.38, (×10⁶/per brain), p=0.01] (FIGS. 3A and 3B), with predominant effects on CD11b⁺CD45^{hi} macrophages/activated microglia (FIG. 3C) that expressed CD74 [vehicle: 64.93±11.62 versus DRα1-MOG-35-55: 33.28±6.64, (×10³/per brain), p=0.03] (FIG. 3D). In contrast, no changes were observed in total numbers or CD74 expression of non-activated CD11b⁺CD45^{int} microglia after treatment with DRα1-MOG-35-55 [vehicle: 17.57±2.81 versus DRα1-MOG-35-55: 11.71±2.31, (×10³/per brain), p=0.12]. These results thus demonstrate that treatment with the DRα1-MOG-35-55 construct can reduce the infiltration and local activation of CD11b⁺CD45^{hi} cells in the injured brain and their expression of CD74 after TBI.

Polarization of CD11b⁺CD45^{int} cells toward a pro-inflammatory phenotype may promote brain injury after TBI. CD86 and CD206 are two markers used to characterize the pro-inflammatory versus anti-inflammatory phenotypes of CD11b⁺CD45^{int} cells, respectively. Therefore, the expression of CD86 and CD206 by brain-infiltrating CD11b⁺CD45^{int}. cells was determined. The results clearly demonstrated a reduction of CD86-expressing inflammatory CD11b⁺CD45^{int} cells [vehicle: 22.96±2.62 versus DRα1-MOG-35-55: 12.44±3.36, ×10³/per brain), p=0.03] and an increase of anti-inflammatory CD206-expressing CD11b⁺CD45^{int} cells after treatment with DRα1-MOG-35-55 [vehicle: 32.03±7.08 versus DRα1-MOG-35-55: 60.09±7.59, ×10³/per brain), p=0.02] (FIG. 3E).

### Example 3—Effects of DRα1-MOG-35-55 Treatment on Peripheral CD11b⁺ Cells

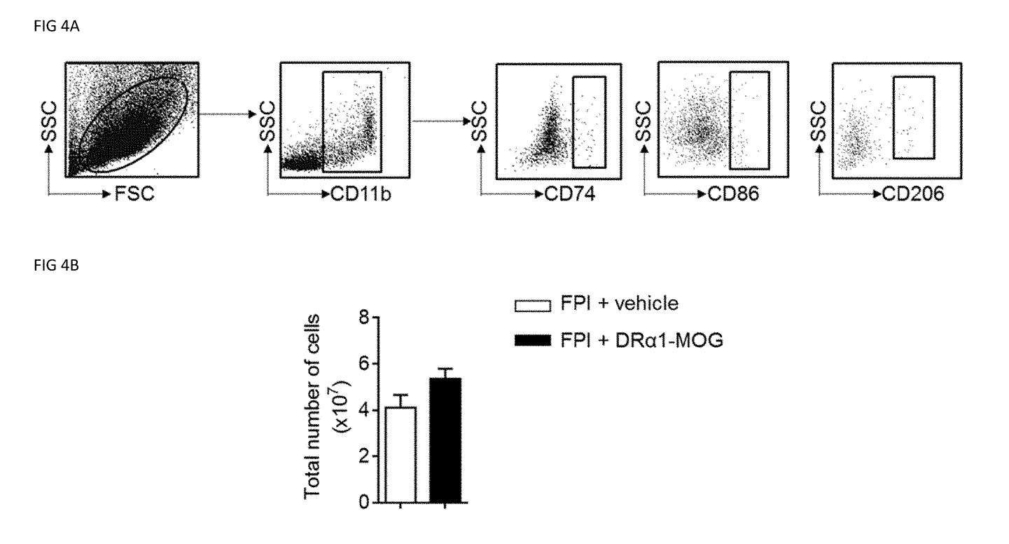

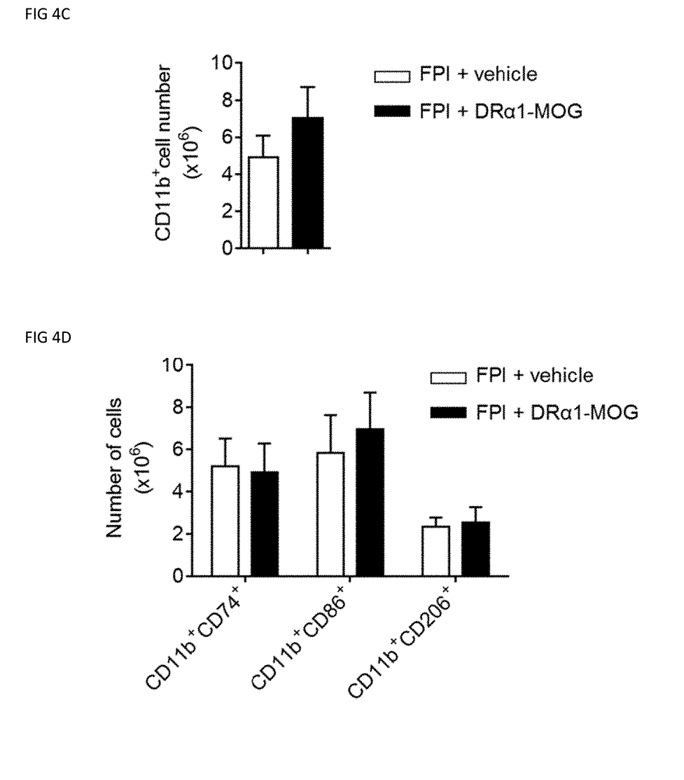

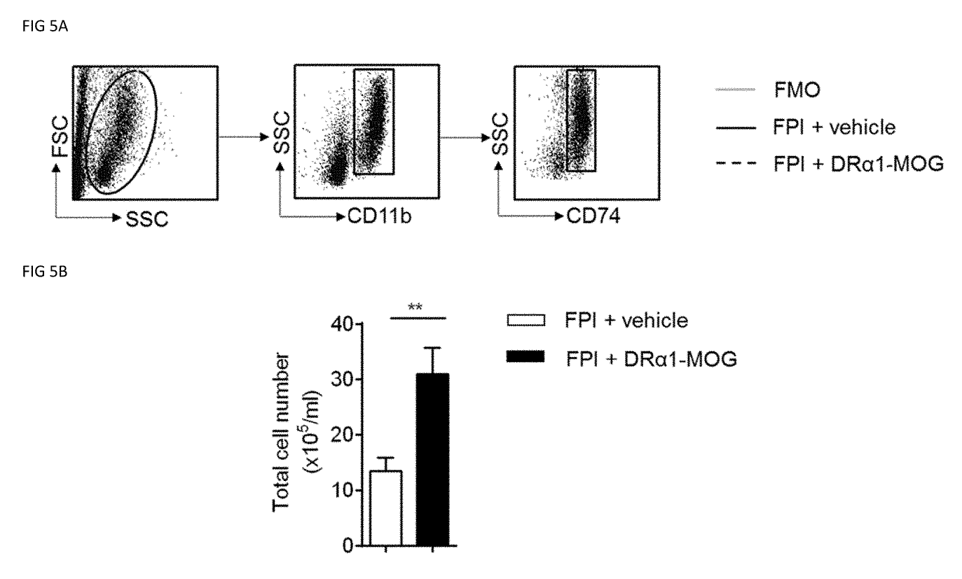

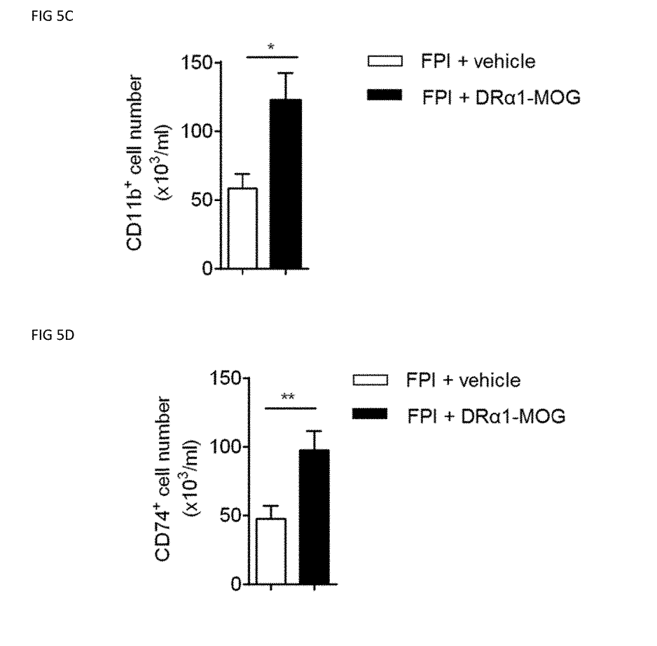

To evaluate the effects of DRα1-MOG-35-55 treatment on peripheral CD11b⁺ cells, the number of CD11b⁺ cells and their expression of CD74 in the spleen or peripheral blood on day 3 after TBI induction were measured. No changes were observed in total [vehicle: 4.11±0.55 and DRα1-MOG-35-55: 5.35±0.43, (×10⁷/per spleen), p=0.11] or CD11b⁺ cell numbers [vehicle: 4.925±1.16 and DRα1-MOG-35-55: 7.06±1.66, (×10⁶/per spleen), p=0.32], or CD74⁺, CD86⁺ or CD206⁺ cells in the spleen after DRα1-MOG-35-55 treatment [CD74, vehicle: 5.21±1.32 versus DRα1-MOG-35-55: 4.92±1.38, (×10⁶/per spleen), p=0.88; CD86, vehicle: 5.84±1.81 versus DRα1-MOG-35-55: 6.98±1.72, (×10⁶/per spleen), p=0.66; CD206, vehicle: 2.36±0.44 versus DRα1-MOG-35-55: 2.56±0.72, (×10⁶/per spleen), p=0.82] (FIGS. 4A-4D). In contrast, treatment with DRα1-MOG-35-55 induced significant increases in total circulating cell numbers as well as in CD11b⁺[vehicle: 58.33±10.47 versus DRα1-MOG-35-55: 122.60±20.05, (×10⁶/ml), p=0.01] and CD74⁺ cells [vehicle: 47.53±9.39 versus DRα1-MOG-35-55: 97.32±14.11, (×10³/ml), p=0.01] in the peripheral blood (FIGS. 5A-5D).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A method of treating traumatic brain injury in a human subject, the method comprising administering to the human subject in need thereof a composition comprising an effective amount of DRα1-MOG-35-55 and a pharmaceutically acceptable carrier.

2. The method of claim 1, further comprising administering the composition to the human subject after the onset of traumatic brain injury.

3. The method of claim 2, wherein the composition is administered to the human subject within 1-24 hours after the onset of the traumatic brain injury.

4. The method of claim 2, wherein the composition is administered to the human subject one or more days after the onset of traumatic brain injury.

5. The method of claim 1 wherein the composition comprises a dose of 1-100 mg/kg of DRα1-MOG-35-55.

6. The method of claim 1 wherein the composition comprises a dose of 1-100 mg of DRα1-MOG-35-55.

7. The method of claim 1 wherein the composition is administered to the subject by subcutaneous or intravenous administration.

8. The method of claim 1 wherein the DRα1-MOG-35-55 comprises an amino acid sequence at least 95% identical to the amino acid sequence of SEQ ID NO: 1.

10. The method of claim 9, wherein the DRα1-MOG-35-55 comprises the amino acid sequence of SEQ ID NO: 1.

11. The method of claim 1 wherein the traumatic brain injury results from a closed head injury.

11. The method of claim 1 wherein the traumatic brain injury results from an open head injury.

11. The method of claim 1 wherein the traumatic brain injury results from a diffuse axonal injury.

11. The method of claim 1 wherein the traumatic brain injury results from a contusion of the brain.

11. The method of claim 1 wherein the traumatic brain injury results from a penetrating trauma to the brain.
